(19) United States
(12) Patent Application Publication
(10) Pub. No.: US 2013/0137126 A1
(43) Pub. Date: May 30, 2013

(54) USE OF A BETA-GLUCOSIDASE ACTIVATOR FOR THE DETECTION AND/OR IDENTIFICATION OF C. DIFFICILE

(71) Applicants: Marie Cellier, Montalieu Vercieu (FR); Diane Halimi, Saint-Maurice-de-Beynost (FR); Sylvain Orenga, Neuville sur Ain (FR)

(72) Inventors: Marie Cellier, Montalieu Vercieu (FR); Diane Halimi, Saint-Maurice-de-Beynost (FR); Sylvain Orenga, Neuville sur Ain (FR)

(73) Assignee: BIOMERIEUX, Marcy l'Etoile (FR)

(21) Appl. No.: 13/816,135

(22) PCT Filed: Aug. 30, 2011
(86) PCT No.: PCT/FR11/51982

§ 371 Date: Feb. 8, 2013

(30) Foreign Application Priority Data
Sep. 1, 2010 (FR) 1056928

(51) Int. Cl. C12Q 1/34

(52) U.S. Cl. CPC C12Q 1/34
USPC 435/18

(57) ABSTRACT

The present invention relates to a reaction medium comprising at least one beta-glucosidase substrate and a compound of the general formula Ar-beta-D-glucoside where Ar- designates an aromatic compound, different from said substrate. According to the invention, such a medium can be employed in a C. difficile detection and/or identification process.

The present invention relates to a process of detecting and/or identifying Clostridium difficile. It also relates to a reaction medium specific to Clostridium difficile comprising a beta-glucosidase activator.

Clostridium difficile (C. difficile) is a Gram-positive bacillus which is strictly anaerobic and spore-forming. It is the cause of intestinal pathologies of variable severity: medium or explosive diarrhoeas, pseudomembranous colitises or toxic megacolon which themselves can be the cause of a sepsis. Clostridium difficile is transmitted by oral ingestion of spores which are resistant to stomach acidity and germinate in the intestine. An imbalance of the commensal flora of the colon enables the proliferation of C. difficile, which produces toxins which are the cause of colitises. In many cases, the imbalance of the commensal flora is the result of taking antibiotics. C. difficile can be considered to be an inoffensive environmental microorganism, which under certain conditions becomes an opportunistic pathogen. The asymptomatic digestive carriage of C. difficile is estimated at 3% in the adult population, but it can reach 15 to 25% of subjects after antibiotic treatment or a stay in a unit with high endemicity. A high carriage level is generally observed, from 20 to 40% in hospitalised patients up to 50-70% in young children. The possibility of early and rapid diagnosis is a key element in the prevention of severe complications, and in effective clinical care.

The diagnosis is based on searching for toxins and on culturing.

The isolation of toxins directly from stools is an excellent marker of the presence of a toxigenic strain of C. difficile. The reference method consists in searching for a cytopathogenic effect (CTA for cytotoxic activity in English) by means of cell culturing. This method is very sensitive but is not standardised and requires specialised technicians and a response time of several days. ELISA-type immunoenzymatic tests have also been developed which detect either toxin A alone, or toxins A and B. The time for obtaining a result is reduced. However, it is recommended to combine immunological techniques used to detect toxins with bacterial culturing in order to isolate the C. difficile strains. This culture can be employed on a C. difficile agar marketed by the Applicant, the C. difficile colonies then being identified by biochemical methods such as the API® 20 A gallery or the rapid ID 32 A gallery. However, the identification by gallery makes it necessary to have a pure culture of the bacteria to be identified, on a suitable growth medium in order to obtain sufficient biomass (3 to 4 McFarland) which takes, at best, 48 h (isolation culture from 24 to 72 hours from sampling, which only gives the presumptive detection, then culturing from 24 to 72 hours to generate sufficient biomass). The reading of the gallery then takes place after 4 h of incubation for rapid ID 32 A, and 24 to 48 h for the 20A gallery.

Finally, the diagnosis can be carried out using molecular biology techniques. However, these techniques are not currently routinely used.

By means of its work with the aim of improving the direct identification of C. difficile, in a bacterial culture, the Applicant demonstrated that the use of beta-glucosidase enzymatic substrate(s) makes it possible to easily and quickly identify C. difficile. Said use of beta-glucosidase substrate is described in application WO 2009/092982 as reducing the time for obtaining a C. difficile identification result.

The early and rapid identification of C. difficile potentially present in a sample remains a prime objective, and the Applicant has pursued its work by more particularly seeking to accelerate the revelation of enzymatic activity. It has thus unexpectedly demonstrated that a reaction medium comprising a beta-glucosidase substrate and a beta-glucosidase activator of the general formula Ar-beta-D-glucopyranoside, where Ar- corresponds to an aromatic ring or to several adjoined aromatic rings, lets C. difficile be rapidly identified in a sample, in other words that such a medium lets the enzymatic activity of at least some C. difficile strains be detected earlier. The use of an Ar-beta-D-glucoside compound lets beta-glucosidase be activated and enables a significantly more intense coloration of the C. difficile colonies to be observed. Advantageously, the Ar-beta-D-glucoside compounds have a C. difficile beta-glucosidase activating power far superior to that of other beta-D-glucoside derivatives such as cellobiose (4-O-beta-D-glucopyranosyl-D-glucose) or Methyl-beta-D-glucose.

It is known that the compounds of the general formula Ar-beta-D-glucoside, where Ar- represents at least one homocyclic or heterocyclic aromatic ring, can be used as beta-glucosidase substrates. This use is reported in application WO 2009/092982 (supra). Hildebrand and Schroth likewise report that arbutin, or hydroquinone beta-D-glucopyranoside, is used as a source of carbon, at a concentration of 0.5% weight/volume, in other words 5 g/l, in combination with glucose, in a reaction medium. The hydrolysis of arbutin in hydroquinone, measured by chromatographic methods, is a marker of the beta-glucosidase activity of Pseudomonas syringae (Hildebrand and Schroth, Applied Microbiology, 1964; 12 (6): 487-491). This discourages the person skilled in the art from using an Ar-beta-D-glucoside compound as a beta-glucosidase activator in a reaction medium for the detection or identification of C. difficile, since it would find itself in competition with the substrate intended to reveal the enzymatic activity of the target microorganism.

Finally, the prior art reports that orally administered arbutin is excreted and metabolised in urines and can, as such, be used for its diuretic and antibacterial properties (Schindler et al., 2002; J. Clin. Pharmacol.; 42 (8): 920-927; Siegers et al., 2003, Phytomedicine; 10 (Suppl 4): 58-60). Such a use also discourages use of arbutin in a reaction medium to detect a bacteria.

Before a detailed description of the invention, the following definitions are given to allow the invention to be better understood. They are in no way limiting.

Reaction medium is understood to be a medium comprising all of the elements necessary for the expression of a metabolism and/or for the growth of microorganisms. The reaction medium may be solid, semi-solid or liquid. Solid medium is understood to be a gelled medium. Agar is the conventional gelling agent in microbiology for culturing microorganisms, but it is possible to use gelatine, agarose or other natural or artificial gelling agents. A number of preparations are commercially available such as, for example, Columbia agar, Trypcase-soy agar, MacConkey agar, Sabouraud agar or more generally those described in the Handbook of Microbiological Media (CRC Press). The reaction medium according to the invention must permit the growth of C. difficile.

The reaction medium may comprise one or more elements in combination, such as amino acids, peptones, carbohydrates, nucleotides, minerals, vitamins, etc. The medium may also comprise a colorant. As an indication, Evans blue, neutral red, sheep's blood, horse's blood, an opacifier such as titanium oxide, nitroaniline, malachite green, brilliant green, one or more metabolic indicators, one or more metabolic regulators, etc. may be cited as colorants.

The reaction medium may be a revealing medium or a culturing and revealing medium. In the first case, the culturing of the microorganisms is carried out prior to seeding and, in the second case, the detection and/or identification medium also constitutes the culture medium.

The person skilled in the art may also use a bi-plate, which makes it possible to easily compare two media, comprising different substrates or different selective mixtures, onto which an identical biological sample has been deposited.

The reaction medium may comprise one or more growth activators of C. difficile strains. Growth activator is understood to be a compound or a group of compounds which stimulates the growth of microorganisms. In particular, blood, serum and egg yolks can be cited. Without being limiting, a concentration of between 0.1 and 10% is particularly suitable for the present invention.

The reaction medium may comprise one or more reducing agents. Reducing agent is understood to be a compound or a group of compounds which facilitates the growth of the anaerobic germs by neutralising the dissolved oxygen present in the medium. In particular, Cysteine, Pyruvate, Oxyrase, Sodium sulfite, Dithionite, Histidine and Ferrous sulphide can be cited. Without being limiting, a concentration of between 0.05 and 50 g/l, preferably between 0.1 and 2 g/l, is particularly suitable for the present invention.

The reaction medium may comprise one or more C. difficile spore germination inducers. Spore germination inducers are understood to be a compound or a group of compounds which promotes the passing of C. difficile from the spore state to the vegetative state. In particular, sodium taurocholate can be cited.

Without being limiting, a concentration of between 0.1 and 10 g/l, preferably between 1 and 5 g/l, is particularly suitable for the present invention.

The reaction medium may comprise one or more selective agents. Selective agent is understood to be any compound able to prevent or slow the growth of a microorganism other than the target microorganism. Without being limiting, a concentration of between 5 mg/l and 5 g/l is particularly suitable for the present invention.

Antibiotics and antifungals can be cited as selective agents. Antibiotics are to be understood as any compound capable of preventing or slowing the growth of a bacterium. In particular, they belong to the groups of cephalosporins, aminoglycosides, polypeptides, sulfamides and quinolones. As an indication, it is in particular possible to cite the antibiotics cefotaxime, ceftazidime, cefoxitin, ceftriaxone, cefpodoxime, aztreonam, gentamicin, Trimethoprim, tobramycin, moxalactam, fosfomycin, D-cycloserine, Polymyxin, Colistin and quinolones such as nalidixic acid.

Antifungals are to be understood as any compound capable of preventing or slowing the growth of a yeast or mould. As an indication, it is possible to cite in particular amphotericin B, fluconazole, itraconazole, voriconazole and cycloheximide.

Generally, the reaction medium can furthermore contain a substrate which makes it possible to detect an enzymatic or metabolic activity of the target microorganisms by means of a signal which can be directly or indirectly detected. For a direct detection, this substrate can be linked to a part which acts as a fluorescent or chromogenic marker. For an indirect detection, the reaction medium according to the invention can further include a pH indicator, which is sensitive to the pH variation which results from the consumption of the substrate and which reveals the growth of the target microorganisms. Said pH indicator can be a chromophore or a fluorophore. Neutral red, aniline blue and bromocresol blue can be cited as examples of chromophores. The fluorophores comprise for example 4-methylumbelliferone, hydroxycoumarin derivatives or resorufin derivatives.

Beta-glucosidase substrate capable of identifying C. difficile is understood to be a substrate which results, under suitable growth conditions, in the coloration of C. difficile. In particular, mention can be made of 2 Hydroxyphenyl-beta glucoside (Catechol-beta glucoside); Magenta-beta glucoside (5 Bromo-6 chloro-3 indoxyl-beta glucoside); DHF-beta glucoside (Dihydroxyflavone-beta glucoside); Aesculin (Aesculetin-beta glucoside); CHE-beta glucoside (3,4 Cyclohexenoaesculetin-beta glucoside); 8 Hydroxiquinoline-beta glucoside; X-beta glucoside (5 Bromo-4 chloro-3 indoxyl-beta glucoside); Rose-beta glucoside (6 Chloro-3 indoxyl-beta glucoside); 6 Bromo-3 indoxyl-beta glucoside; Blue-beta glucoside (5 Bromo-3 indoxyl-b glucoside); 6 Fluoro-3 indoxyl-beta glucoside; Alizarin-beta glucoside; (P) Nitrophenyl-beta glucoside; 4 Methylumbelliferyl-beta glucoside, Naphtholbenzein-beta glucoside, Indoxyl-N methyl-beta glucoside, 5 Bromo-4 chloro-3 indoxyl-N methyl-beta glucoside, Naphthyl-beta glucoside; Aminophenyl-beta glucoside; Dichloroaminophenyl-beta glucoside, Aldol™-beta-glucoside (BIOSYNTH, Lukas M. Wick, Alexander Bayer, Christophe Weymuth, Günter Schabert, Vanessa Pfister, Aysel Aslan, Urs P. Spitz, “Novel Chromogenic Enzyme Substrates”, ASM General Meeting, 2009, Philadelphia, I-091/285).

The reaction medium may further comprise an enzymatic activator. An enzymatic activator is understood to be a compound which acts to activate the enzymatic digestion process, in other words to reduce the incubation time for revealing the enzymatic activity and/or to increase said enzymatic activity.

The beta-glucosidase activators according to the invention have as a general formula Ar-beta-D-glucoside, where Ar- represents an aromatic ring. By aromatic ring we mean an unsaturated compound, i.e. having double bonds and including one or more adjoined homoatomic or heteroatomic rings, each ring having 5 to 7 summits. In a non-limiting manner, mention can be made of arbutin (hydroquinone-beta-D-glucoside), 4-methylumbelliferyl-beta-glucoside, 4 aminophenyl-beta-glucoside, salicin (2-(hydroxymethyl)-phenyl-beta-D-glucoside), and aesculin (6,7-dihydroxy-coumarin-beta-glucoside). Some of these compounds are beta-glucosidase substrates. In terms of the invention, they are considered as activators because they make it possible to reduce the incubation time necessary for the detection of hydrolysis of the compound used as a substrate, or they make it possible to increase this hydrolysis. Thus surprisingly, the combination of an enzymatic substrate and an activator according to the invention makes it possible to have an earlier and/or more intense detection of the enzymatic activity than when these compounds are used separately.

The activating power of a compound may notably be demonstrated for certain strains of C. difficile which have a belated and/or weak beta-glucosidase activity, if the combination of said compound and the substrate enables earlier and/or more intense detection than if the substrate of activator was used only at a molarity at least equivalent to the sum of the molarities of the substrate and of the activator in combination.

Biological sample is intended to mean a small part or small quantity isolated from an entity for analysis. This can be a clinical sample, derived from a specimen of biological fluid, or a food sample, derived from any type of food. This sample may thus be liquid or solid. Mention may be made, in a non-limiting manner, of a clinical sample of total blood, serum, plasma, urines or faeces sample, nose, throat, skin, wound or cerebrospinal fluid, a food sample from water, from drinks such as milk or a fruit juice, from yogurt, from meat, from eggs, from vegetables, from mayonnaise, from cheese; from fish, etc., a food sample derived from an animal feed, such as, in particular, a sample derived from bone meal. This specimen may be used as it is or may, prior to the analysis, undergo a preparation of enrichment, extraction, concentration or purification type, according to the methods known to the person skilled in the art.

After contacting the sample, the reaction medium is incubated at an appropriate temperature, generally of between 20 and 50° C., preferably between 30 and 40° C. Preferably, the incubation is carried out in anaerobiosis, in other words in the absence of oxygen, in accordance with the techniques known to the person skilled in the art.

The invention therefore relates to a reaction medium comprising at least one beta-glucosidase substrate and a compound of the general formula Ar-beta-D-glucoside, where Ar- designates an aromatic compound, different from said substrate, at a concentration lower than 5 g/l.

The compound Ar-beta-D-glucoside is preferably at a concentration of between 0.3 and 1.5 g/I.

The compound of formula Ar-beta-D-glucoside is preferably selected from amongst arbutin or hydroquinone-beta-glucoside, 4-methylumbelliferyl-beta-glucoside, 4-aminophenyl-beta-glucoside, salicin or 2-(hydroxymethyl)phenyl-beta-D-glucoside, aesculin or 6,7-dihydroxy-coumarin-beta-glucoside.

The compound Ar-beta-D-glucoside is preferably arbutin.

According to a preferred embodiment of the invention, the beta-glucosidase substrate is selected from amongst Alizarin-beta-glucoside, Magenta-beta-glucoside (5-Bromo-6-chloro-3-indoxyl-beta-glucoside), DHF-beta-glucoside, 3HF-beta-glucoside and CHE-beta-glucoside (3,4-Cyclohexenoesculetine-beta-glucoside) and the ALDOL™ beta-glucosides by the company BIOSYNTH (Wick et al. 2009. ASM General meeting—Philadelphia (Pa., USA)). Said beta-glucosidase substrate is at a concentration of between 25 and 1,000 mg/l, preferably between 50 and 400 mg/I.

According to the invention, the enzymatic activator is an enzymatic substrate of which the hydrolysis product is different from the hydrolysis product of the enzymatic substrate.

According to a preferred embodiment of the invention, the enzymatic activator is an enzymatic substrate of which the product cannot be detected in the reaction medium by the naked eye, whereas the product of the hydrolysis of the enzymatic substrate can be.

According to another preferred embodiment of the invention, the reaction medium further comprises a C. difficile spore germination activator, said activator preferably being sodium taurocholate, at a concentration of between 0.1 and 10.0 g/l, preferably of between 1.0 and 5.0 g/l.

The invention relates to a process of detecting and/or identifying C. difficile comprising the steps consisting of:
a) placing a sample likely to contain C. difficile in contact with a reaction medium comprising at least one beta-glucosidase substrate and a compound of the general formula Ar-beta-D-glucoside, different from said substrate, where Ar- designates an aromatic compound;
b) incubating, and
c) detecting the hydrolysis of the beta-glucosidase substrate, which indicates the presence of C. difficile.

According to a preferred embodiment, the process according to the invention employs in step a) a medium such as defined supra.

According to another preferred embodiment, the incubation employed in step b) of the process according to the invention is performed in anaerobiosis.

The invention relates to the use of a compound of the general formula Ar-beta-D-glucoside as a beta-glucosidase activator in a reaction medium.

Preferably, said compound is at a concentration lower than 5 g/l, preferably of between 0.3 and 1.5 g/l. Preferably, said compound is chosen from: arbutin or hydroquinone-beta-glucoside, 4-methylumbelliferyl-beta-glucoside, 4-aminophenyl-beta-glucoside, salicin or 2-(hydroxymethyl)phenyl-beta-D-glucoside, aesculin or 6,7-dihydroxy-coumarin-beta-glucoside. More preferably, said compound is arbutin.

Preferably, said reaction medium is a medium for detecting and/or identifying C. difficile.

The examples below are given by way of explanation and are in no way limiting. They will allow the invention to be better understood.

### EXAMPLE 1

### Demonstrating the Activating Effect of Arbutin on a Beta-Glucosidase Substrate

1. Medium and Microorganisms

Eight strains of Clostridium difficile, from the Applicant's collection, were tested on media comprising arbutin at different concentrations with or without CHE-beta-glucoside present. The reading of the dishes is then carried out at 24 h. The media is made up as follows:

C. difficile ChromID® base common to all of the media:

| Compounds | Conc. in g/l |
|---|---|
| Peptone, tissue and cell extracts | 12.5 |
| Sodium chloride | 6 |
| Magnesium sulphate | 0.3 |
| L-arginine | 1 |
| Sodium bisulfite | 0.1 |
| agar | 13 |
| Ammonium iron citrate | 0.3 |
| Glucose | 0.2 |
| TRIS | 0.1 |
| Sodium taurocholate | 2.5 |

Then addition of arbutin and/or CHE-beta-glucoside, according to the media:

| Concen- | Me- | me- | me- | me- | me- | me- | | |
|---|---|---|---|---|---|---|---|---|
| tration | dium | dium | dium | dium | dium | dium | medium | medium |
| in g/l | T1 | A1 | A2 | A3 | T2 | B1 | B2 | B3 |
| CHE-b- | 0 | 0 | 0 | 0 | 0.3 | 0.3 | 0.3 | 0.3 |
| glucoside | | | | | | | | |
| Arbutin | 0 | 0.1 | 0.3 | 1 | 0 | 0.1 | 0.3 | 1 |
| g/l | | | | | | | | |

The references and the respective ribotypes of the strains tested are indicated in the following table:

| No. of the strains | Ribotype |
|---|---|
| 06 03 006 | O27 |
| 08 01 092 | O27 |
| 08 01 104 | O27 |
| 08 01 109 | O27 |
| 08 01 112 | O103 |
| 08 01 113 | O27 |
| 08 01 116 | O27 |
| 08 01 118 | O27 |

2. Test

The media are distributed into Petri dishes.

The seeding is performed from 48 h pre-cultures at 37° C. in anaerobiosis.

A suspension in physiological water at 0.5 McF is carried out and then the strains are seeded with the 10 μl calibrated loop.

Readings are performed after 24 h of incubation at 37° C. in anaerobiosis.

3. Results

Reading scale of the enzymatic activity/ coloration
0=no activity
0.5=very pale coloration
1=clear low-intensity coloration
2=distinct medium-intensity coloration,
3=presence of an intense coloration,
4=presence of a very intense coloration.

The coloration obtained with this substrate is grey up to an intensity of 1, and black for the other values.

| | | T1 | A1 | A2 | A3 | T2 | B1 | B2 | B3 |
|---|---|---|---|---|---|---|---|---|---|
| Clostridium difficile | 24 h | 0 | 0 | 0 | 0 | 1 | 1 | 4 | 4 |
| 109 | | | | | | | | | |
| Clostridium difficile | 24 h | 0 | 0 | 0 | 0 | 0.5 | 1 | 4 | 4 |
| 104 | | | | | | | | | |
| Clostridium difficile | 24 h | 0 | 0 | 0 | 0 | 0 | 1 | 4 | 4 |
| 092 | | | | | | | | | |
| Clostridium difficile | 24 h | 0 | 0 | 0 | 0 | 1 | 1 | 4 | 4 |
| 006 (ATCC O27) | | | | | | | | | |
| Clostridium difficile | 24 h | 0 | 0 | 0 | 0 | 1 | 1 | 4 | 4 |
| 116 | | | | | | | | | |
| Clostridium difficile | 24 h | 0 | 0 | 0 | 0 | 0.5 | 1 | 3 | 4 |
| 118 | | | | | | | | | |
| Clostridium difficile | 24 h | 0 | 0 | 0 | 0 | 1 | 1 | 4 | 4 |
| 113 | | | | | | | | | |
| Clostridium difficile | 24 h | 0 | 0 | 0 | 0 | 0 | 4 | 4 | 4 |
| 112 | | | | | | | | | |

Thus, after 24 h of incubation, we obtained: in number of strains coloured in black on the different media:

| | T1 | A1 | A2 | A3 | T2 | B1 | B2 | B3 |
|---|---|---|---|---|---|---|---|---|
| Intensity of | 0/8 | 0/8 | 0/8 | 0/8 | 4/8 | 8/8 | 8/8 | 8/8 |
| Coloration >=1 | | | | | | | | |

4. Interpretation

In the absence of substrate, no coloration is observed.

Arbutin alone does not allow coloration of the colonies to be obtained either.

Conversely, upon the addition of arbutin in the presence of CHE-beta-glucoside, an activation of the beta-glucosidase is noted, which is expressed by an intensification of coloration (grey to black).

5. Conclusion:

Arbitin acts as a beta-glucosidase activator on the Clostridium difficile strains.

### EXAMPLE 2

### Test of an Optimal Concentration of Arbutin on Different C. Difficile Strain

1. Medium and Microorganisms

Twenty-four strains of Clostridium difficile, from the Applicant's collection, were tested on 5 media each comprising a different arbutin concentration. The reading of the dishes is then carried out at 24 h. The media used are prepared on the basis of C. difficile chromID® medium indicated supra, for example 1, to which are added cefotaxime at 0.016 g/l, fungizone at 0.005 g/l and D-cycloserine at 0.25 g/l (final concentrations), and arbutin according to the following table:

| | medium 1 | medium 2 | medium 3 | medium 4 | medium 5 |
|---|---|---|---|---|---|
| Arbutin g/L | 0 | 0.75 | 1 | 1.25 | 1.5 |

The references and the respective ribotypes of the strains tested are indicated in the following table:

| No. of the strains | Ribotype |
|---|---|
| 02 01 042 | ND |
| 02 01 043 | ND |
| 02 06 146 | ND |
| 02 06 148 | ND |
| 06 03 006 | 027 |
| 08 01 078 | 027 |
| 08 01 080 | O27 |
| 08 01 084 | O27 |
| 08 01 088 | 010 |
| 08 01 092 | 027 |
| 08 01 094 | 078 |
| 08 01 095 | 027 |
| 08 01 096 | 027 |
| 08 01 098 | 027 |
| 08 01 100 | 027 |
| 08 01 102 | 001 |
| 08 01 103 | 014 |
| 08 01 104 | 027 |
| 08 01 109 | 027 |
| 08 01 111 | 027 |
| 08 01 113 | 027 |
| 08 01 114 | 002 |
| 08 01 116 | 027 |
| 08 01 118 | 027 |

ND = not determined

2. Tests

The media are distributed into Petri dishes.

The seeding is performed from 48 h pre-cultures at 37° C. in anaerobiosis.

A suspension in physiological water at 0.5 McF is carried out and then the strains are seeded with the 10 μl calibrated loop.

Readings are performed after 24 h of incubation in anaerobiosis.

3. Results

Reading scale of the enzymatic activity/ coloration
0=no activity
0.5=very pale coloration
1=clear low-intensity coloration
2=distinct medium-intensity coloration,
3=presence of an intense coloration,
4=presence of a very intense coloration.

The coloration obtained with this substrate is grey up to an intensity of 1, and black for the other values.

The results noted here, on the different media, were obtained after 24 h of incubation at 37° C. in anaerobiosis.

| | M1 | M2 | M3 | M4 | M5 |
|---|---|---|---|---|---|
| Clostridium difficile | 3 | 2.5 | 2.5 | 2.5 | 2.5 |
| 42 | | | | | |
| Clostridium difficile | 3 | 3 | 3 | 3 | 3 |
| 43 | | | | | |
| Clostridium difficile | 0 | 2 | 2 | 1.5 | 1.5 |
| 146 | | | | | |
| Clostridium difficile | 3 | 3 | 3 | 3 | 3 |
| 148 | | | | | |
| Clostridium difficile 6 | 0.75 | 3 | 3 | 3 | 3 |
| Clostridium difficile | 3 | 3 | 3 | 3 | 3 |
| 78 | | | | | |
| Clostridium difficile | 0.75 | 1.5 | 3 | 3 | 2.5 |
| 80 | | | | | |
| Clostridium difficile | 2 | 2 | 2 | 2 | 2 |
| 84 | | | | | |
| Clostridium difficile | 4 | 4 | 4 | 4 | 4 |
| 88 | | | | | |
| Clostridium difficile | 0 | 3 | 3 | 2.75 | 2.75 |
| 92 | | | | | |
| Clostridium difficile | 3 | 3 | 3 | 3 | 2.75 |
| 94 | | | | | |
| Clostridium difficile | 0.5 | 3 | 3 | 2.5 | 1 |
| 95 | | | | | |
| Clostridium difficile | 1 | 2.5 | 3 | 3 | 3 |
| 96 | | | | | |
| Clostridium difficile | 0.75 | 3 | 3 | 3 | 3 |
| 98 | | | | | |
| Clostridium difficile | 0.5 | 3 | 3 | 3 | 2.75 |
| 100 | | | | | |
| Clostridium difficile | 3 | 3 | 3 | 3 | 3 |
| 102 | | | | | |
| Clostridium difficile | 3 | 4 | 4 | 4 | 4 |
| 103 | | | | | |
| Clostridium difficile | 0.1 | 2.5 | 3 | 3 | 3 |
| 104 | | | | | |
| Clostridium difficile | 0.1 | 3 | 3 | 3 | 3 |
| 109 | | | | | |
| Clostridium difficile | 0 | 2.5 | 2.5 | 2.5 | 3 |
| 111 | | | | | |
| Clostridium difficile | 0 | 2.5 | 3 | 3 | 3 |
| 113 | | | | | |
| Clostridium difficile | 3 | 3 | 3 | 2.5 | 2.5 |
| 114 | | | | | |
| Clostridium difficile | 0 | 2.5 | 2.5 | 2.5 | 2.5 |
| 116 | | | | | |
| Clostridium difficile | 0 | 2.5 | 2.5 | 2.5 | 2.5 |
| 118 | | | | | |

Thus, after 24 h of incubation, we obtained the following, in number of black-coloured strains, on the different media:

| | | | | Medium | Medium |
|---|---|---|---|---|---|
| | Medium 1 | Medium 2 | Medium 3 | 4 | 5 |
| Coloration >= 1 | 12/24 | 24/24 | 24/24 | 24/24 | 24/24 |
| | of which | of which | of which | of | of |
| | 0/15 O27 | 15/15 O27 | 15/15 O27 | which | which |
| | | | | 15/15 | 15/15 |
| | | | | O27 | O27 |

4. Interpretation

Upon the addition of arbutin into the medium, the coloration is clearly improved. At 24 h, on the medium without arbutin, only 12 strains out of 24 have a coloration greater than or equal to 1, whereas in the presence of 750 mg/L of arbutin, 24 strains /24 are colored with a minimum intensity of 1.

The strains which already have a strong coloration on the control seem to be very little impacted, or unimpacted by the presence of arbutin if 1 g/L is not exceeded. Beyond that, a slight reduction in the coloration intensity is noted for certain strains.

5. Conclusion

Arbutin certainly acts here as a beta-glucosidase activator, and its action seems significant for the early detection of Clostridium difficile and in particular for the ribotype O27.

### EXAMPLE 3

### Test for the Activating Effect of Arbutin on Various Beta-Glucosidase Substrates

1. Medium and Microorganisms

The eight strains of Clostridium difficile tested in Example 1 were tested on media comprising DHF-beta-glucoside (media A), alizarin-beta-glucoside (media B) or 3HF-beta-glucoside (Media C) and an increasing concentration of arbutin.

The reading of the dishes is then carried out at 24 h and 48 h.

The media used are prepared on the basis of the C. difficile chromID® medium indicated supra, for example 1, to which arbutin is added according to the following table:

| | Medium T | Medium 1 | medium 2 | medium 3 |
|---|---|---|---|---|
| Arbutin g/L | 0 | 0.250 | 0.5 | 1 |

According to the substrates, the media are therefore as follows:
DHF-beta-glucoside at 400 mg/L (media TA, 1A, 2A and 3A)
Alizarin-beta-glucoside at 50 mg/L (media TB, 1 B, 2B and 3B)
3HF-beta-glucoside at 300 mg/L (media TC, 1C, 2C and 3C)

2. Tests

The media are distributed into Petri dishes.

The seeding is performed from 48 h pre-cultures at 37° C. in anaerobiosis.

A suspension in physiological water at 0.5 McF is carried out and then the strains are seeded with the 10 μl calibrated loop.

Readings are performed after 24 h and 48 h of incubation at 37° C. in anaerobiosis.

3. Results

Reading scale of the enzymatic activity/ coloration
0=no activity
0.5=very pale coloration
1=clear low-intensity coloration
2=distinct medium-intensity coloration,
3=presence of an intense coloration,
4=presence of a very intense coloration.

Colour of the Colonies:

Medium A: Brown

Medium B: Mauve

Medium C: Brown

| | | DHF-b- | | | | | | | | | | | |
|---|---|---|---|---|---|---|---|---|---|---|---|---|---|
| | | glucoside | | | | Alizarin-b-glucoside | | | | 3HF-b-glucoside | | | |
| | | TA | 1A | 2A | 3A | TB | 1B | 2B | 3B | TC | 1C | 2C | 3C |
| Clostridium difficile | 24 h | 2 | 2.5 | 3 | 3.5 | 0.5 | 1 | 1 | 0.75 | 0 | 0 | 0 | 0 |
| 6 | 48 h | 2.5 | 2.5 | 3 | 3.5 | 0.5 | 1.5 | 1.5 | 1.5 | 0.5 | 0.5 | 1 | 1.5 |
| Clostridium difficile | 24 h | 2 | 2 | 3 | 3.5 | 0.5 | 1 | 1 | 0.75 | 0 | 0 | 0 | 0 |
| 92 | 48 h | 2.5 | 2.5 | 3 | 3.5 | 0.5 | 1.5 | 1.5 | 1.5 | 0.5 | 1 | 1.5 | 1.5 |
| Clostridium difficile | 24 h | 2 | 2.5 | 3 | 4 | 0.5 | 1 | 1 | 0.75 | 0.5 | 0.5 | 0.5 | 0.5 |
| 104 | 48 h | 2.5 | 2.5 | 3 | 4 | 0.5 | 1.5 | 1.5 | 1.5 | 1 | 1 | 1.5 | 2 |
| Clostridium difficile | 24 h | 2 | 2.5 | 3 | 4 | 0.5 | 1 | 1 | 1 | 0 | 0 | 0 | 0 |
| 109 | 48 h | 2.5 | 3 | 3 | 4 | 0.5 | 1.5 | 1 | 1 | 1 | 1.5 | 1.5 | 1.5 |
| Clostridium difficile | 24 h | 2 | 2 | 2 | 2 | 1 | 1 | 1 | 1 | 0 | 0 | 0 | 0 |
| 112 | 48 h | 3 | 3 | 3 | 2.5 | 1.5 | 1.5 | 1.5 | 1.5 | 0 | 1 | 1.5 | 1.5 |
| Clostridium difficile | 24 h | 2 | 2 | 2 | 2.5 | 1 | 1 | 1 | 1 | 0 | 0 | 0 | 0 |
| 113 | 48 h | 2.5 | 2.5 | 3 | 3 | 1 | 2 | 2 | 1.5 | 1 | 1 | 1.5 | 2 |
| Clostridium difficile | 24 h | 2 | 2 | 2 | 2.5 | 1 | 1.25 | 1.25 | 1 | 0.1 | 0.1 | 0.1 | 0.1 |
| 116 | 48 h | 2.5 | 2.5 | 2.5 | 3 | 1 | 1.5 | 1 | 1 | 0.5 | 1.5 | 1.5 | 1.5 |
| Clostridium difficile | 24 h | 2 | 2 | 2 | 3 | 1 | 1.5 | 1.5 | 1 | 0 | 0 | 0 | 0 |
| 118 | 48 h | 2.5 | 2.5 | 3 | 3 | 1.5 | 2 | 2 | 1.5 | 0.5 | 1 | 1.5 | 1.5 |

4. Interpretation

In the presence of arbutin, an improvement in the coloration intensity is noted regardless of the substrate used. This improvement is above all visible during the use of DHF-β-glucoside, from 24 h onward and primarily in the presence of 1 g/l of arbutin. For alizarin-beta-glucoside, the improvement is less distinct at 24 h, but is more marked at 48 h on all of the media containing arbutin. Finally, for 3HF-beta-glucoside the effect of this compound is only visible at 48 h, but a real activation of beta-glucosidase activity is noted regardless of the concentration used and regardless of what the strains are.

However, the ideal concentration varies according to the substrate used, and would be near 1 g/l with DHF-beta-glucoside and 3HF-beta-glucoside, and 500 mg/l with alizarin-beta-glucoside.

5. Conclusion:

This test demonstrates the importance of arbutin for the activation of beta-glucosidase activity in strains of Clostridium difficile.

### EXAMPLE 4

### Test for the Activating Effect of Ar-Beta-Glucoside Compounds on Beta-Glucosidase Activity

1. Medium and Microorganisms

Six strains of Clostridium difficile were tested on the media comprising a compound which could act as a beta-glucosidase activator in these strains. The ribotypes of these strains are indicated in the tables supra. That of the strain bearing the reference 08 01 091 is 056. The compounds tested as beta-glucosidase activators are as follows:
Arbutin;
O-methyl b-glucoside;
Cellobiose;
Salicin;
4 Aminophenyl-b-glucoside;
4-methyl-umbelliferone-b-glucoside;
aesculin.

They were tested at concentrations which make it possible to have the same molarity for each of the compounds.

The media used are prepared on the basis of the C. difficile chromID® medium indicated supra, for example 1, to which is added the CHE-beta-glucoside substrate at 0.3 g/l and, respectively, one of the compounds according to the following table:

| | | O- | | | | | |
|---|---|---|---|---|---|---|---|
| | | methyl | Cello- | Sali- | | 4MU- | |
| | Arbutin | b-Glu | biose | cin | 4AminoPhe | b-glu | aesculin |
| T | — | — | — | — | — | | |
| M1 | 1 g/l | — | — | — | — | | |
| M2 | — | 0.7 g/l | — | — | — | | |
| M3 | — | — | 1.25 g/l | — | — | | |
| M4 | — | — | — | 1 g/l | — | | |
| M5 | — | — | — | — | 1 g/l | | |
| M6 | | | | | | 1.25 g/l | |
| M7 | | | | | | | 1.25 g/l |

2. Tests

The media are distributed into Petri dishes.

The seeding is performed from 48 h pre-cultures at 37° C. in anaerobiosis.

A suspension in physiological water at 0.5 McF is carried out and then the strains are seeded with the 10 μl calibrated loop.

Readings are performed after 24 h of incubation at 37° C. and in anaerobiosis.

3. Results

Reading scale of the enzymatic activity/ coloration
0=no activity
0.5=very pale coloration
1=clear low-intensity coloration
2=distinct medium-intensity coloration,
3=presence of an intense coloration,
4=presence of a very intense coloration.

The coloration obtained with this substrate is grey up to an intensity of 1, and black for the other values.

| | T | M1 | M2 | M3 | M4 | M5 | M6 | M7 |
|---|---|---|---|---|---|---|---|---|
| Clostridium difficile | 4 | 4 | 0 | 4 | 4 | 4 | 4 | 4 |
| 91 | | | | | | | | |
| Clostridium difficile | 0.5 | 3 | 0 | 0.5 | 4 | 4 | 3 | 3 |
| 94 | | | | | | | | |
| Clostridium difficile* 6 | 4/1 | 4 | 0 | 4/1 | 4 | 4 | 3 | 3/1 |
| Clostridium difficile | 0.5 | 4 | 0 | 0 | 4 | 3 | 3 | 3 |
| 111 | | | | | | | | |
| Clostridium difficile | 0 | 4 | 0 | 0 | 4 | 4 | 4 | 4 |
| 112 | | | | | | | | |
| Clostridium difficile | 0.5 | 3 | 0 | 0.5 | 4 | 4 | 3 | 3 |
| 116 | | | | | | | | |

*= strain displaying colonies of heterogeneous colours;
the results indicated in the form of a fraction x/y signify that certain strains display an intensity x and others display an intensity y.

4. Interpretation

O-methyl-beta-glucoside and cellobiose do not have an activating effect on beta-glucosidase of Clostridium difficile. On the contrary, a reduction in the coloration is noted on the strains displaying a coloration on the control.

Conversely, as for arbutin, there is an observed activation of beta-glucosidase and therefore an increase of the coloration of the colonies in the presence of salicin, 4-Aminophenyl-beta-glucoside, 4-methyl-umbelliferone or aesculin. A homogenisation of the coloration is also noted for the heterogeneous strains, in the presence of one of these compounds. For the medium containing aesculin, a strong diffusion of black coloration is noted in the agar, which sometimes makes it difficult to read the medium.

5. Conclusion:

This test demonstrates the activating effect of beta-glucosidase of C. difficile by compounds possessing at least one aromatic ring such as arbutin, salicin, 4-Aminophenyl-beta-glucoside, 4-methyl-umbelliferone or aesculin.

### EXAMPLE 5

### Test of 2 Substrates of Beta-Glucosidase Based on ALDOL™ (ALDOL-198 455-Beta-Glucoside and ALDOL™484-Beta-Glucoside—BIOSYNTH)

1. Medium and Microorganisms

Eight strains of Clostridium difficile were tested on 4 different media, respectively a control growth medium (T), a medium (1) containing 75 mg/L of ALDOL™ 455-beta-glucoside, a medium (2) containing 75 mg/L of ALDOL™ 484-beta-glucoside, and a medium (3) containing CHE-b-glucoside at a level of 300 mg/L and ammonium iron citrate (300 mg/L).

The composition of the nutrient base common to all of the media is indicated below. It is the C. difficile chromID® base without ammonium iron citrate, but supplemented with 1 g/L of arbutin.

| Compounds | Conc. in g/l |
|---|---|
| Tissue and cell extracts | 12.5 |
| Sodium chloride | 6 |
| Magnesium sulphate | 0.3 |
| L-arginine | 1 |
| Sodium bisulfite | 0.1 |
| Agar | 13 |
| Arbutin | 1 |
| Glucose | 0.2 |
| TRIS | 0.1 |
| Sodium taurocholate | 2.5 |

After the agars have melted, the base is separated into 4*80 mL, distributed into flasks T, 1, 2 and 3. 300 mg/L of CHE-beta-glucoside and 300 mg/L of ammonium iron citrate will have been weighed into flask 3 in advance. An autoclaving cycle sterilises the media. After the media are returned to supercooling, the ALDOL™-based substrates are added as additives, solubilised in a solvent such as DMSO (dimethylsulfoxide), from a concentrated stock solution.

2. Tests

The media are distributed into Petri dishes.

The seeding is performed from 48 h pre-cultures at 37° C. in anaerobiosis.

A suspension in physiological water at 0.5 McF is carried out and then the strains are seeded with the 10 μl calibrated loop.

Readings are performed after 24 h of incubation at 37° C. and in anaerobiosis. The results are listed hereafter.

3. Results

Following the enzymatic hydrolysis of various substrates by the strains of C. difficile, the colorations of the isolated colonies obtained on the media 1, 2 and 3 are respectively yellow, orange and brown.

Reading scale of the expression of enzymatic activity (coloration intensities)
0=no activity
0.5=very pale coloration
1=clear low-intensity coloration
2=distinct medium-intensity coloration,
3=presence of an intense coloration,
4=presence of a very intense coloration.

| | | T | 1 | 2 | 3 |
|---|---|---|---|---|---|
| Clostridium difficile | 24 h | 0 | 3 | 4 | 2 |
| 0801108 | | | | | |
| Clostridium difficile | 24 h | 0 | 3 | 3 | 4 |
| 0801105 | | | | | |
| Clostridium difficile | 24 h | 0 | 3 | 4 | 4 |
| 0801088 | | | | | |
| Clostridium difficile | 24 h | 0 | 3 | 4 | 3 |
| 0801091 | | | | | |
| Clostridium difficile | 24 h | 0 | 3 | 3 | 3 |
| 0801094 | | | | | |
| Clostridium difficile | 24 h | 0 | 2 | 3 | 2 |
| 0603006 | | | | | |
| Clostridium difficile | 24 h | 0 | 3 | 3 | 4 |
| 0801112 | | | | | |
| Clostridium difficile | 24 h | 0 | 3 | 3 | 4 |
| 0801116 | | | | | |

4. Interpretation

The ALDOL™ 455-beta-glucoside and ALDOL™484-beta-glucoside (BIOSYNTH) substrates enable an excellent detection of the C. difficile. The coloration intensities (yellow and orange respectively) obtained are high, from 24 h of incubation. The performances obtained with these two substrates are substantially identical to those obtained with CHE-beta-glucoside.

1. A reaction medium comprising at least one beta-glucosidase substrate and a compound of the formula Ar-beta-D-glucoside where Ar- designates an aromatic compound, different from said substrate, at a concentration lower than 5 g/l.

2. The reaction medium according to claim 1, wherein the compound Ar-beta-D-glucoside is at a concentration of between 0.3 and 1.5 g/l.

3. The reaction medium according to claim 1, wherein the compound Ar-beta-D-glucoside is selected from the group consisting of hydroquinone-beta-D-glucopyranoside, 4-methylumbelliferyl-beta-glucoside, 4-aminophenyl-beta-glucoside, salicin or 2-(hydroxymethyl)phenyl-beta-D-glucoside, and aesculin or 6,7-dihydroxy-coumarin-beta-glucoside.

4. The reaction medium according to claim 1, wherein the compound Ar-beta-D-glucoside is arbutin.

5. The reaction medium according to claim 1, wherein the beta-glucosidase substrate is selected from the group consisting of Alizarin-beta-glucoside, Magenta-beta-glucoside (5-Bromo-6-chloro-3-indoxyl-beta-glucoside), DHF-beta-glucoside, 3HF-beta-glucoside and CHE-beta-glucoside (3,4-Cyclohexenoesculetine-beta-glucoside) and ALDOL™ beta-glucoside.

6. The reaction medium according to claim 1, wherein the beta-glucosidase substrate is at a concentration of between 25 and 1000 mg/l.

7. The reaction medium according to claim 1, further comprising a Clostridium difficile spore germination activator.

8. The reaction medium according to claim 7, wherein said Clostridium difficile spore germination activator is sodium taurocholate.

9. The reaction medium according to claim 8, wherein sodium taurocholate is at a concentration of between 0.1 and 10 g/l.

10. A process of detecting and/or identifying Clostridium difficile comprising:
a) placing a sample likely to contain Clostridium difficile in contact with a reaction medium comprising at least one beta-glucosidase substrate and a compound of the formula Ar-beta-D-glucoside, different from said substrate, where Ar- designates an aromatic compound;
b) incubating the sample: and
c) detecting the hydrolysis of the beta-glucosidase substrate when Clostridium difficile is present in the sample.

11. (canceled)

12. The process according to claim 10, wherein the incubation employed in step b) is performed in anaerobiosis.

13. (canceled)

14. (canceled)

15. The process according to claim 10, wherein the compound Ar-beta-D-glucoside is at a concentration of between 0.3 and 1.5 g/l.

16. The process according to claim 10, wherein the compound Ar-beta-D-glucoside is selected from the group consisting of hydroquinone-beta-D-glucopyranoside, 4-methylumbelliferyl-beta-glucoside, 4-aminophenyl-beta-glucoside, salicin or 2-(hydroxymethyl)phenyl-beta-D-glucoside, and aesculin or 6,7-dihydroxy-coumarin-beta-glucoside.

17. The process according to claim 10, wherein the compound Ar-beta-D-glucoside is arbutin.

18. The process according to claim 10, wherein the beta-glucosidase substrate is selected from the group consisting of Alizarin-beta-glucoside, Magenta-beta-glucoside (5-Bromo-6-chloro-3-indoxyl-beta-glucoside), DHF-beta-glucoside, 3HF-beta-glucoside and CHE-beta-glucoside (3,4-Cyclohexenoesculetine-beta-glucoside) and ALDOL™ beta-glucoside.

19. The process according to claim 10, wherein the beta-glucosidase substrate is at a concentration of between 25 and 1000 mg/l.

20. The process according to claim 10, wherein the reaction medium further comprises a Clostridium difficile spore germination activator.

21. The process according to claim 20, wherein said Clostridium difficile spore germination activator is sodium taurocholate.

22. The process according to claim 21, wherein sodium taurocholate is at a concentration of between 0.1 and 10 g/l.
